(19) United States
(12) Patent Application Publication
(10) Pub. No.: US 2019/0321213 A1
(43) Pub. Date: Oct. 24, 2019

(54) Adjustable Waterproof Barrier For An Ostomy Appliance

(71) Applicant: Robert Allen Morrison, SR., Seminole, FL (US)

(72) Inventor: Robert Allen Morrison, SR., Seminole, FL (US)

(21) Appl. No.: 15/960,429

(22) Filed: Apr. 23, 2018

(51) Int. Cl. A61F 5/449; A61F 5/448

(52) U.S. Cl. CPC A61F 5/449; A61F 5/448

(57) ABSTRACT

A novel method for bathing with a waterproof barrier covering an ostomy appliance. The removable barrier is a bowl having a removable plate with an opening configured to receive a folded ostomy pouch. The flange of the bowl is in contact with the user's skin surrounding the ostomy appliance. A belt having a first end located opposite a second end is connected to the bowl. The first end and the second end of the belt are fastened around the abdomen of the user. The opening of the bowl forms a chamber. The chamber is configured to retain the ostomy appliance when the top portion and the lower portion of the ostomy appliance are folded over and secured with a fastener. The belt can be adjusted to tighten or loosen the bowl that is positioned over an ostomy appliance of a user to create a water tight seal.

### BACKGROUND OF THE INVENTION

### 1. Field of the Invention

This invention relates, generally, to a removable barrier for an ostomy appliance. More particularly, it relates to a method of using an adjustable container that is positioned over both an ostomy wafer and an ostomy pouch to create a water proof barrier.

### 2. Background Art

An ostomy is a surgical procedure performed to create an artificial opening, often referred to as a stoma, that allows bodily waste to pass through the stoma in the abdomen into a prosthetic including, but not limited to, a pouch or an ostomy bag that is connected to the outside of the body. Essentially, bodily waste is rerouted from its normal path because of malfunctioning parts of the urinary or digestive system. This change in the way urine or stool naturally exits the body can be lifesaving to many of individuals and patients enduring disease or injury including, but not limited to, birth defects, cancer, inflammatory bowel disease, diverticulitis, or incontinence which may necessitate an ostomy surgery. There are many different types of ostomy. In a first example, the stoma located on the abdomen of a person can be connected with the bottom of the small intestine to bypass the colon, rectum and anus, which is referred to as ileostomy. In a second example, the stoma can be connected to the colon to bypass the rectum and anus, which is referred to as colostomy. In a third example, a urostomy is a surgical procedure that creates a stoma for the urinary system.

For most ostomies, a pouch is worn over the stoma to collect stool or urine. An ostomy appliance refers to a prosthesis which is a mechanical replacement for a biological function. Many ostomy appliances in today's market have a collection pouch that is attached mechanically or with an adhesive to a mounting plate, including, but not limited to, a flange, a wafer, or a baseplate. Currently, an ostomy wafer has an adhesive boarder on a first side that adheres to a user's skin when the wafer is applied to the area around the stoma. The ostomy wafer protects and shields the skin around the stoma from solid waste exposure which may cause skin irritation or infection.

Bathing is often a concern for people living with an ostomy. A problem arises when a user is showering or swimming with the ostomy appliance connected to their abdomen in that the adhesion of the pouch or the adhesion of the ostomy wafer becomes compromised. As a result, the adhesive may become sticky and the adhesive of the ostomy wafer may lose its bond with the user's skin or the adhesive of the pouch may lose its bond with the ostomy wafer. The ostomy appliance may be loosened or even fall off completely due to the exposure of the ostomy appliance to water and heat associated with bathing. Further, the excessive time taken to dry the ostomy appliance after bathing can be both inconvenient and time consuming for the ostomy appliance user. As a result of bathing, the wear time of the ostomy appliance may be shorter than anticipated. Thus, there is a need to provide a waterproof cover for the ostomy appliance to extend the wear time of the ostomy appliance.

Ostomy covers that are presently manufactured have a stoma opening. Although these covers substantially enclose an ostomy appliance, they are not waterproof because the stoma opening of the pouch exposes the adhesive of the ostomy appliance to water when the user is showering or swimming. As a result, the ostomy appliance may lose adhesion and fall off of the desired attachment site, rendering the ostomy appliance inoperable for its intended purpose. Additionally, if the ostomy appliance falls off of the user when the user is swimming, the body of water may become contaminated with fecal matter or the fecal matter may contact the user's skin and cause irritation or infection. Thus, it would be more desirable to have a waterproof ostomy appliance cover capable of covering an ostomy appliance without the need of a stoma opening.

Many users, such as individuals having had an ostomy surgery have been known to fold the ostomy pouch over and affix the pouch in a folded orientation with a piece of tape. A user then will use a shower cap to cover the ostomy appliance. A user may even use a layer of plastic wrap in an attempt to create a barrier between the water exposure and the ostomy appliance. Costs associated with the tape and plastic wrap is costly and the waste generated as a result of the use of the tape and plastic wrap is not environmentally friendly. Further, the tape used often leaves a sticky residue on a user's skin which is undesirable. Thus, there is a need for an ostomy appliance cover that eliminates the need for tape and eliminates the need for plastic wrap.

However, in view of the prior art considered as a whole at the time the present invention was made, it was not obvious to those of ordinary skill in the pertinent art how the identified needs could be fulfilled.

### SUMMARY OF THE INVENTION

The long-standing but heretofore unfulfilled need for a novel method for bathing with a waterproof barrier covering an ostomy appliance. The removable barrier is a bowl having a flange. The flange of the bowl boarders the opening of the bowl. The flange of the bowl is in contact with the user's skin surrounding the ostomy appliance. A belt having a first end located opposite a second end is connected to the bowl. The first end and the second end of the belt are fastened around the abdomen of the user. The opening of the bowl forms a chamber. The chamber is configured to retain the ostomy appliance when the top portion and the lower portion of the ostomy appliance are folded over and secured with a fastener. The belt can be adjusted to tighten or loosen the bowl that is positioned over an ostomy appliance of a user to create a water tight seal which also includes improvements that overcome the limitations of prior art ostomy appliance covers is now met by a new, useful, and non-obvious invention.

The novel method for bathing with a waterproof barrier covering an ostomy appliance includes the steps of providing a user having a stoma. Next, a waterproof barrier is provided. In a preferred embodiment, the barrier is a bowl having a flange. The bowl has an opening forming a chamber. The flange of the bowl boarders an opening of the bowl. The bowl is connected to a belt. The belt is both removable from the bowl and is adjustable around the abdomen of a user. The belt has a first end located opposite a second end. The first end of the belt is removably connected to the second end of the belt to allow for installation and removal of the novel waterproof barrier to or from the abdomen of a user, respectively.

The bowl has at least one retaining structure protruding from an end of the bowl. The retaining structure is located on an end of the bowl that is located opposite of the chamber opening of the bowl. At least one retaining structure has an opening receiving at least a portion of the adjustable belt. The belt may be tightened of loosened around the abdomen of a user to tighten the bowl to create a waterproof seal around the ostomy appliance. The waterproof seal is capable of being impenetrable by water when a user is bathing. Bathing includes, but is not limited to, swimming or showering.

In the next step of the novel method for bathing with a waterproof barrier covering an ostomy appliance includes the step of providing an ostomy appliance. The ostomy appliance has an ostomy wafer. The ostomy wafer has an opening. The ostomy appliance has an ostomy pouch. The ostomy pouch has an opening. The ostomy pouch has a first fold line located at a top portion of the ostomy pouch. The ostomy pouch has a second fold line located at a bottom portion of the ostomy pouch. Providing a pair of fold lines on an ostomy pouch. It is within the scope of this invention for the folded lines to be a first fold line or a second fold line. The folded lines can be surface indicia indicating to a user where to fold the pouch. The folded lines can further be made of a conformable material such as a thin piece of metal that is capable of maintaining a folded orientation of the ostomy pouch if the user folds the ostomy pouch at the fold line. The bottom portion of the ostomy pouch is folded over at the second fold line and the top portion of the ostomy pouch is folded over at said first fold line. The pair of fold lines aid in the user folding the pouch so that the pouch is a smaller size. This smaller ostomy pouch size is more desirable when a user is bathing because the smaller ostomy pouch can be more easily received by the chamber opening of the novel waterproof barrier.

In the next step of the novel method for bathing with a waterproof barrier covering an ostomy appliance includes the step of orienting the opening of the ostomy wafer with the stoma of a user. Next, an ostomy wafer is adhered to a user's abdomen and the opening of the ostomy wafer is positioned in alignment with the stoma of a user. It is within the scope of this invention for a user to include, but not be limited to, any individual or patient having an abdominal stoma, using the novel ostomy appliance, and using the novel removable barrier to perform the steps of bathing with the novel waterproof barrier covering the novel ostomy appliance.

In the next step of the novel method for bathing with a waterproof barrier covering an ostomy appliance includes the step of orienting the opening of the ostomy pouch in alignment with the opening of the ostomy wafer. The ostomy pouch is then connected to the ostomy wafer. The chamber of the bowl is oriented or placed over the ostomy appliance. Now that the ostomy pouch has been folded at the pair of fold lines, the ostomy pouch is a size small enough to be received by the chamber opening of the waterproof barrier. The waterproof barrier can fully enclose or retain the folded ostomy appliance when the flange of the bowl is connected to or pressed against the skin of a user's abdomen. The waterproof barrier is kept in place on the abdomen of a user with a belt. The first end of the belt is removably connected with the second end of the belt. The first end of the belt and the second end of the belt are fastened around the abdomen of the user. The tightness of the belt can be adjusted to create a water tight seal. This method completely blocks any water from entering the chamber of the barrier from under the flange of the barrier so that neither the ostomy wafer nor the ostomy pouch loses their adhesive connectivity.

In an alternate embodiment, the method for bathing with a waterproof barrier covering an ostomy appliance, further includes providing an ostomy pouch having a fastener connected thereto. The second portion of the ostomy pouch is folded over at the second fold line. The first portion of the ostomy pouch is folded over at the first fold line. A fastener of the top portion of the ostomy pouch is positioned or oriented over the bottom portion of the folded ostomy pouch. The top portion of the ostomy pouch is fastened to the bottom portion of the ostomy pouch to keep the ostomy pouch at a minimal size to be received by the chamber of the waterproof barrier.

### BRIEF DESCRIPTION OF THE DRAWINGS

For a fuller understanding of the nature and objects of the invention, reference should be made to the following detailed description, taken in connection with the accompanying drawings, in which:

FIG. 1 is a perspective view of the novel ostomy pouch having a pair of fold lines and being in communication with a stoma of a user;

FIG. 2 is a perspective view of the bottom portion of the ostomy pouch being folded at a second fold line;

FIG. 3 is a perspective view of the top portion of the ostomy pouch being folded at a first fold line;

FIG. 4 is a perspective view of the fastener of the top portion of the ostomy pouch being connected to the bottom portion of the ostomy pouch when the bottom portion of the ostomy pouch is folded at a second fold line and when the top portion of the ostomy pouch is folded at a first fold line;

FIG. 5 is a front perspective view of the novel waterproof barrier having a pair of retaining structures each having an opening receiving a belt;

FIG. 6 is a side perspective view of the novel waterproof barrier having a retaining structure having an opening receiving a belt;

FIG. 7 is a rear perspective view of the novel waterproof barrier being a bowl having a flange, an opening forming a chamber, and the flange of the bowl bordering an opening of the bowl;

FIG. 8 is an exploded view of the novel waterproof barrier oriented over ostomy pouch, ostomy wafer, and stoma; and,

FIG. 9 is a side cut-away view of the novel waterproof barrier oriented over ostomy pouch, ostomy wafer, and stoma and being connected to the abdomen of a user with an adjustable belt.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

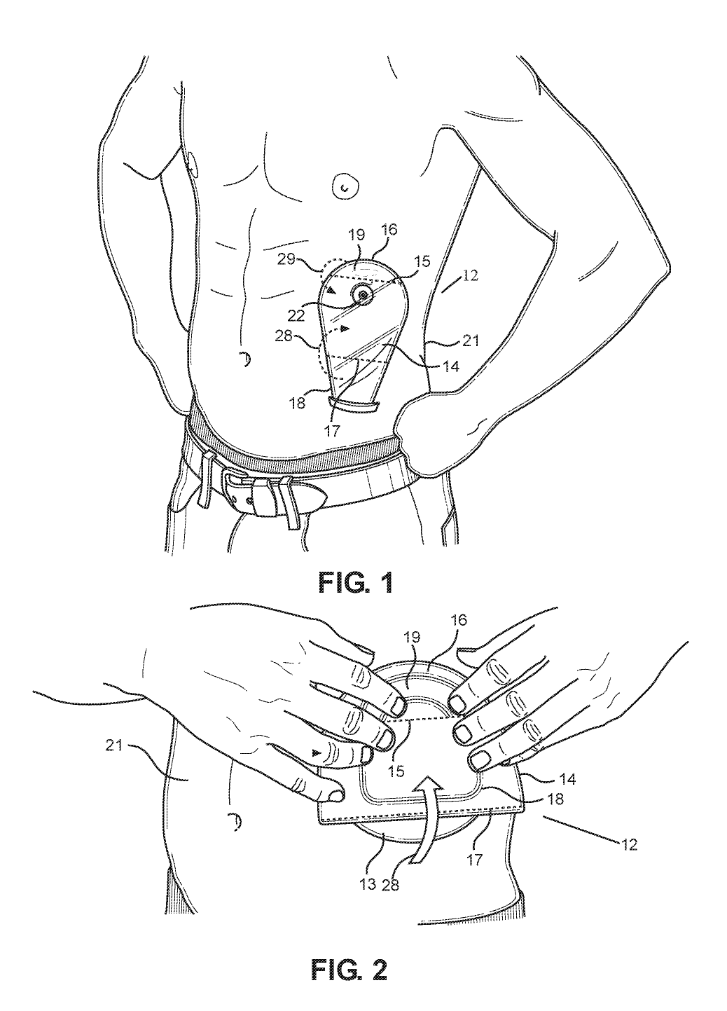

FIG. 1 illustrates a general embodiment of ostomy appliance 12 being in communication with stoma 22 of user's abdomen 21. Ostomy appliance 12 has an ostomy wafer 13 (FIGS. 2-4 and 8-9). Ostomy appliance 12 has ostomy pouch 14. Ostomy pouch 14 has second fold line 17 located at bottom portion 18 of ostomy pouch 14. Ostomy pouch 14 has first fold line 15 locate at top portion 16 of ostomy pouch 14. Top portion 16 of ostomy pouch 14 can have fastener 19 connected there to. It is within the scope of this invention for fastener 19 to include, but not be limited to adhesive, a sticker, a button, a latch, a clasp, or hook and loop attachments. Fold lines 15 and 17 of ostomy pouch 14 can be surface indicia indicating to a user where to fold ostomy pouch 14. Pair of fold lines 15 and 17 can further be made of a conformable material such as a thin piece of metal such as stainless steel that is capable of maintaining a folded orientation 28 and 29 of ostomy pouch 14 if the user folds ostomy pouch 14 at pair of fold lines 17 and 15 respectively.

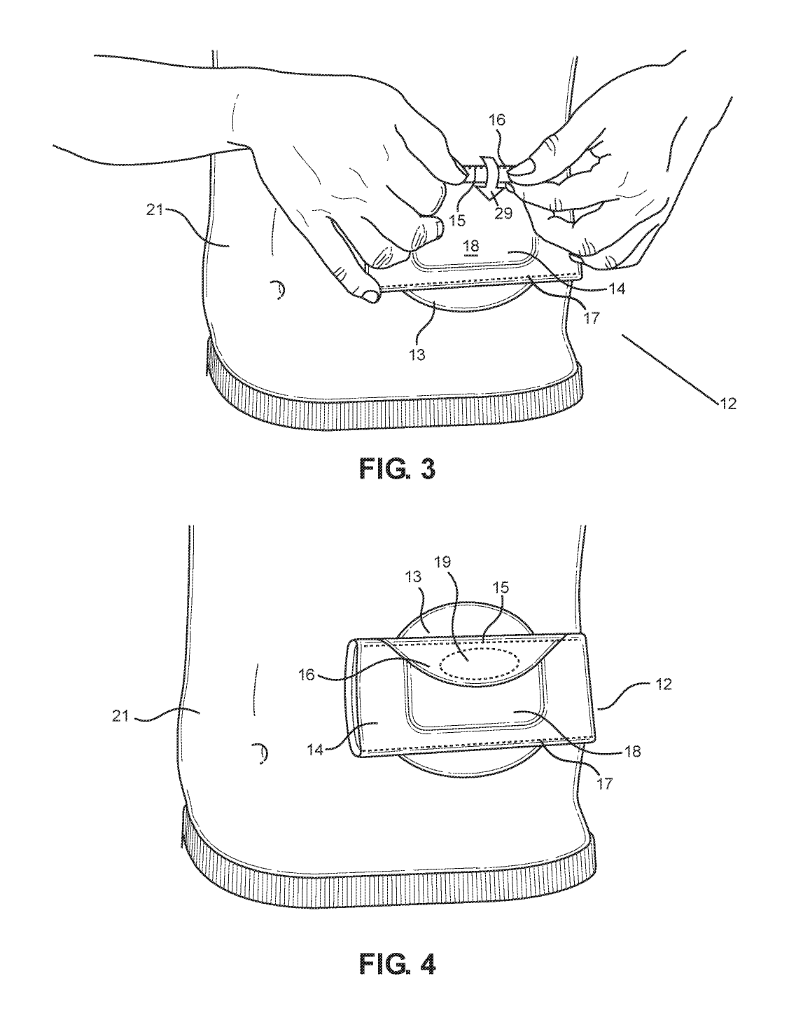

FIG. 2 illustrates ostomy appliance 12 connected to abdomen 21 of a user. Bottom portion 18 of ostomy pouch 14 is folded over 28 at second fold line 17. Top portion 16 of ostomy pouch 14 has first fold line 15 and fastener 19. FIG. 3 depicts the ostomy appliance 12 of FIGS. 1-2 having a top portion 16 of ostomy pouch 14 being folded over 29 at first fold line 15. Top portion 16 of ostomy pouch 14 overlays bottom portion 18 when bottom portion 18 is oriented in a folded configuration.

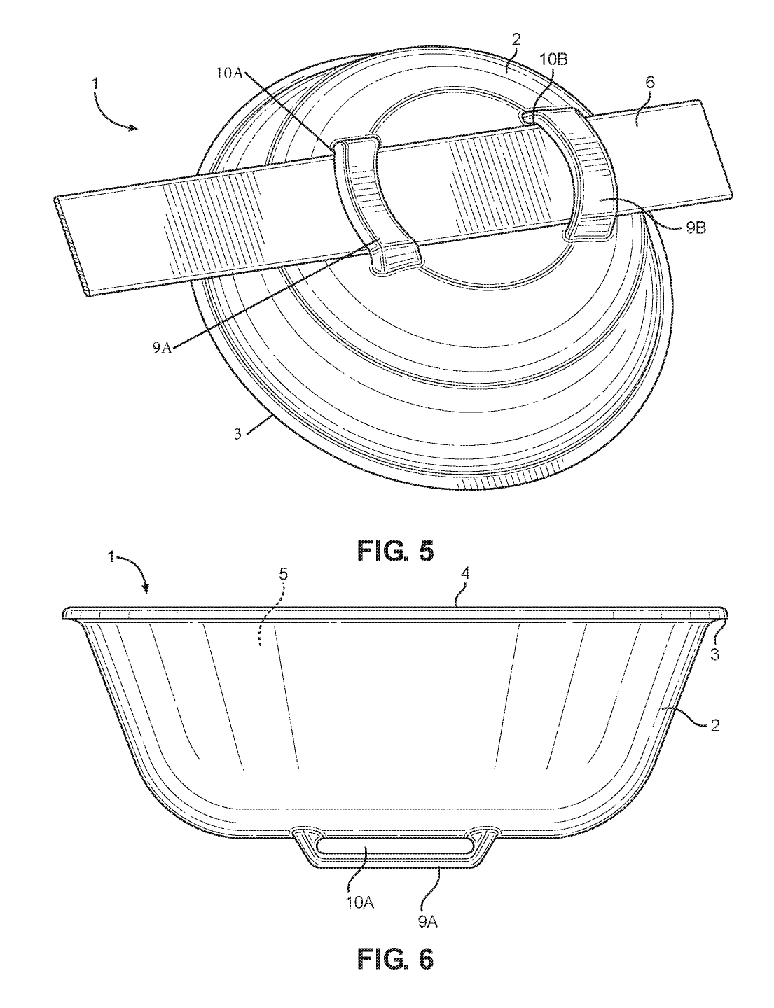

FIG. 4 illustrates ostomy appliance 12 of FIGS. 1-3 having fastener 19 connected to top portion 16 of ostomy pouch 14. Fastener 19 is positioned or oriented over bottom portion 18 of folded 28 ostomy pouch 14. Top portion 16 of ostomy pouch 14 is fastened to bottom portion 18 of ostomy pouch 14 to keep ostomy pouch 14 at a minimal size to be received by chamber 5 of the waterproof barrier 1 (FIGS. 5-9).

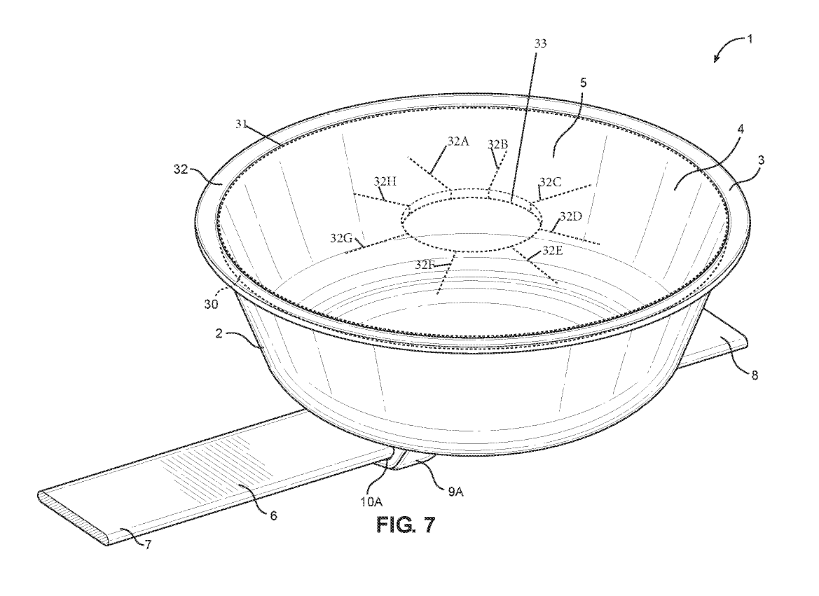

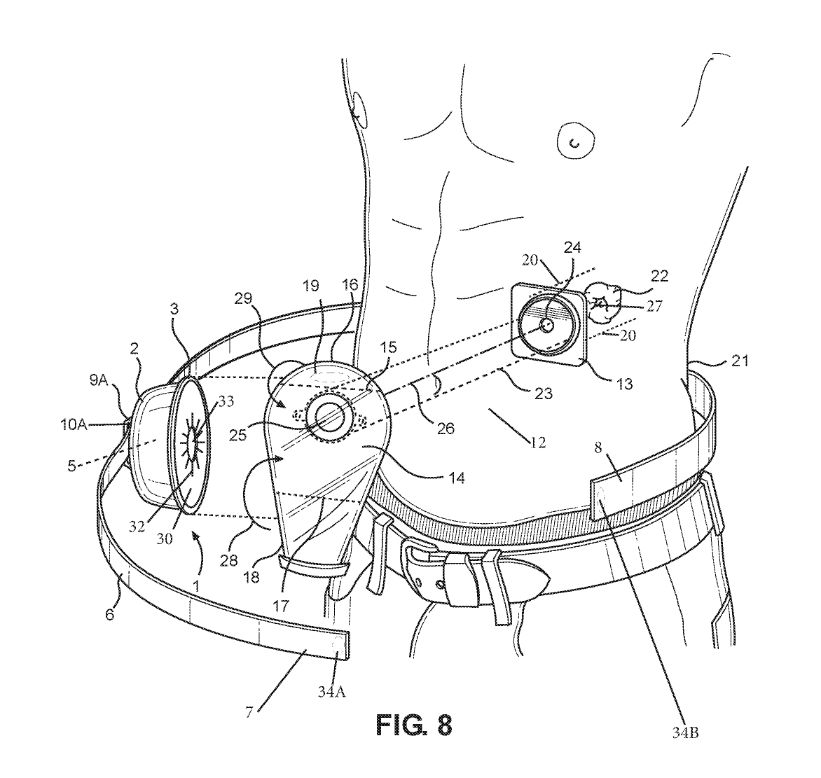

FIGS. 5-9 show waterproof barrier 1 being provided. Barrier 1 has a substantial bowl 2 shape having flange 3. Barrier 1 is bowl 2. Bowl 2 has opening 4 (FIGS. 6-7) forming chamber 5 (FIGS. 6-9). Bowl 2 has flange 3 bordering opening 4 of bowl 2. Bowl 2 is connected to belt 6. Belt 6 is both removable from bowl 2 and is adjustable around the abdomen 21 of a user. FIG. 7 best illustrates belt 6 having first end 7 located opposite second end 8. The first end 7 of belt 6 is removably connected to second end 8 of belt 6 to allow for installation and removal of the novel waterproof barrier 1 to or from the abdomen 21 of a user, respectively. FIG. 8 best illustrates first end 7 of belt 6 can have first fastener 34A connected thereto and second end 8 of belt 6 can have a second fastener 34B connected thereto. In an example, it is within the scope of this current invention for belt 6 ends 7-8 to be tied together, looped together, or buckled together with a buckle, clasp, hook and loop fasteners, or button. In a preferred embodiment, belt 6 is made of a water proof material including, but not limited to, plastic or silicon-plastic.

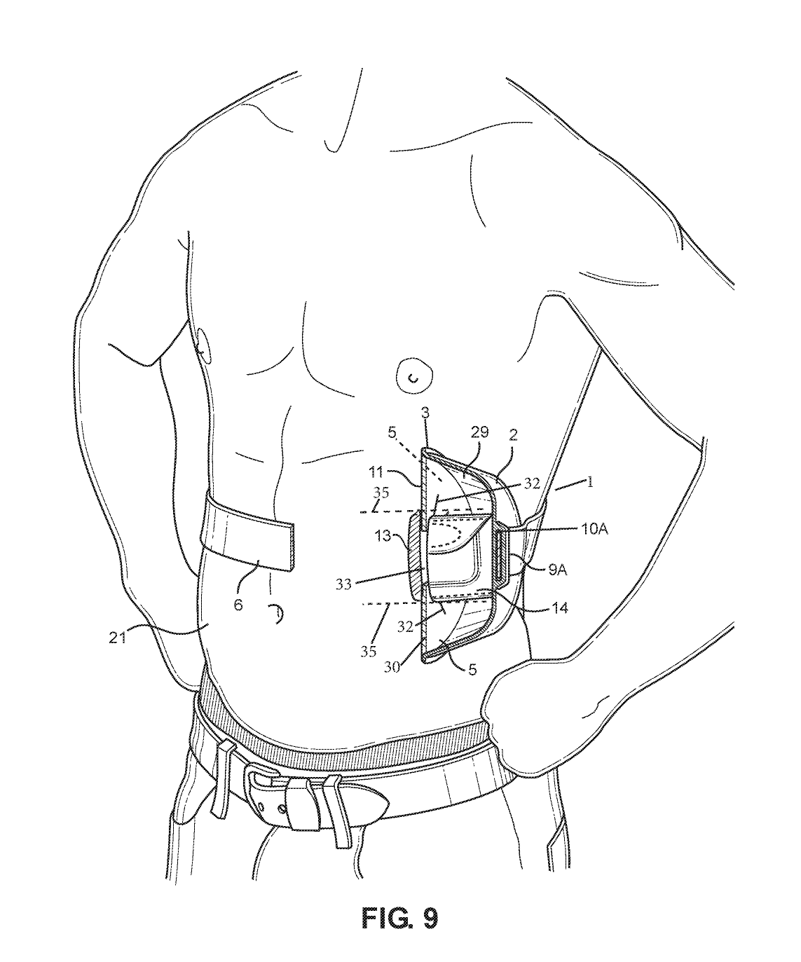

FIGS. 5-9 further illustrate bowl 2 having at least one retaining structure 9A-9B protruding from an end of bowl 2. Retaining structure 9A-9B is located on an end of bowl 2 that is located opposite chamber 5 opening 4 of bowl 2. At least one retaining structure 9A-9B has opening 10A-10B receiving at least a portion of adjustable belt 6. Belt 6 may be tightened of loosened around the abdomen 21 of a user to tighten bowl 2 to create a waterproof seal 11 (FIG. 9) around ostomy appliance 12. Both the ostomy pouch 14 and ostomy wafer 13 are completely covered by bowl 2 when waterproof barrier 1 is installed on user's abdomen 21. The waterproof seal 11 (FIG. 9) is capable of being impenetrable by water when a user is bathing. Bathing includes, but is not limited to, any water submersion, any water exposure, swimming, or showering.

FIG. 8 best shows ostomy wafer 13 having opening 24. Ostomy appliance 12 has ostomy pouch 14 of FIGS. 1-4 having opening 25. Ostomy pouch 14 has first fold line 15 located at top portion 16 of ostomy pouch 14. Ostomy pouch 14 has second fold line 17 located at bottom portion 18 of ostomy pouch 14. Fold lines 15 and 17 on ostomy pouch 14 can be surface indicia indicating to a user where to fold ostomy pouch 14. In an alternate embodiment, pair of fold lines 15 and 17 can further be made of a conformable material such as a thin piece of stainless steel metal that is capable of maintaining a folded orientation 29 and 28 of ostomy pouch 14 if the user folds ostomy pouch 14 at pair of fold lines 15 and 17 respectively. In an alternate embodiment, it is also within the scope of this invention for the ostomy pouch 14 to be rolled (not shown) and then inserted into an ostomy cover (not shown) to minimize the size of the ostomy pouch so that it can more easily fit within bowl 2.

FIGS. 8-9 illustrate the novel method for bathing with waterproof barrier 1 covering ostomy appliance 12 to create a waterproof seal 11 (FIG. 9) including the steps of providing a user 21 having a stoma 22 (FIG. 8). FIG. 8 best shows the next steps of the novel method for bathing with a waterproof barrier covering an ostomy appliance includes the step of orienting 27 the opening 24 of ostomy wafer 13 with stoma 22 of a user. Next, ostomy wafer 13 is adhered to 20 user's abdomen 21 and opening 24 of ostomy wafer 13 is positioned in alignment 27 with stoma 22 of a user. In the next step of the novel method for bathing with a waterproof barrier covering an ostomy appliance includes the step of orienting opening 25 of ostomy pouch 14 in alignment 26 with opening 24 of ostomy wafer 13. Ostomy pouch 14 is then connected to 23 ostomy wafer 13.

FIGS. 7-9 illustrate another embodiment in which bowl 2 of waterproof barrier 1 of the first embodiment includes a removable plate 30 that is received by opening 4 of chamber 5 of bowl 2. In another step, removable plate 30 is provided. Removable plate 30 is removably connected 31 (FIG. 7) to bowl 2 and is positioned to substantially cover opening 4 of chamber 5 of bowl 2. Removable plate 30 is flush with flange 3 when removable plate 30 is inserted into opening 4 of chamber 5 of bowl 2. Removable plate 30 is made of a pliable material including, but not limited to plastic or rubber. Removable plate 30 has central opening 33 configured to receive ostomy pouch 14 (FIGS. 8-9).

Removable plate 30 has plurality of reliefs 32A-32G (FIG. 7) radiating from its central opening 33 circumference to fit varying sized ostomy pouches 14. Central opening 33 located on removable plate 30 receives ostomy pouch 14. Plurality of reliefs 32A-32G (FIG. 7) are located on removable plate 30 around the circumference of central opening 33. When ostomy pouch 14 is received by central opening 33 of removable plate 30, the plurality of reliefs 32A-32G accommodates various sized ostomy pouches 14 and eliminates the need for ostomy pouch 14 to be placed into any additional ostomy covers prior to being inserted into waterproof barrier 1. Plurality of reliefs 32A-32G allow portions or at least one portion of pliable plate 30 to be bendable or depressed so that central opening 33 of plate 30 can be adjusted to be greater in size to receive differing sized ostomy pouches 14, a folded ostomy pouch, or a rolled ostomy pouch. The step of depressing at least a portion of removable plate 30 on at least one relief 32 allows clearance of ostomy pouch 14 to be positioned through central opening 33 of removable plate 30. Ostomy pouch 14 can be rolled up by a user or folded and then inserted into chamber 5 of bowl 2 through opening 33 of removable plate 30.

FIG. 9 illustrates removable plate 30 is positioned between ostomy wafer 13 and ostomy pouch 14 of FIGS. 1-4, when removable barrier 1 is installed on a user's abdomen 21. Ostomy pouch 14 is connected to ostomy wafer 13 and ostomy pouch 14 is retained within chamber 5 of bowl 2. Flange 3 of bowl 2 surrounds ostomy wafer 13 and creates water resistant seal 11 against user's abdomen 21 when installed ion users abdomen 21. Ostomy pouch 14 is inserted through 35 opening 33 of removable plate 30.

It will thus be seen that the objects set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

Now that the invention has been described,

1. A method for bathing with a waterproof barrier covering an ostomy appliance, comprising the steps of:
providing a waterproof barrier, said waterproof barrier is a bowl having a flange, said bowl having an opening forming a chamber, said flange of said bowl boarders said opening of said bowl, said bowl is connected to a belt, said belt having a first end located opposite a second end, said first end of said belt is removably connected to said second end of said belt, said bowl having at least one retaining structure protruding from an end of said bowl, said at least one retaining structure having an opening receiving at least a portion of said belt;
providing an ostomy appliance, said ostomy appliance having an ostomy wafer, said ostomy wafer having an opening, said ostomy appliance having an ostomy pouch, said ostomy pouch having an opening;
providing a removable plate, said removable plate is received by said opening of said chamber of said bowl, said removable plate is removably connected to said bowl, said removable plate is positioned to cover said opening of said chamber of said bowl, said removable plate having a central opening having a circumference, said removable plate having a plurality of reliefs radiating from said circumference of said central opening, whereby, an end of each of said plurality of reliefs abut said central opening;
depressing at least a portion of said removable plate on at least one relief of said plurality of reliefs to allow clearance of said ostomy pouch to be positioned through said central opening of said removable plate;
orienting said opening of said ostomy wafer with a stoma of a user;
adhering said ostomy wafer to a user's abdomen and around said stoma of said user;
orienting said opening of said ostomy pouch in alignment with said opening of said ostomy wafer;
connecting said ostomy pouch to said ostomy wafer;
orienting said chamber of said bowl over said ostomy appliance;
contacting said flange of said bowl with the skin of a user;
connecting said first end of said belt with said second end of said belt, said first end of said belt and said second end of said belt are fastened around an abdomen of said user; and,
adjusting said tightness of said belt to create a water tight seal.

2. The method for bathing with a waterproof barrier covering an ostomy appliance of claim 1, further comprising the steps of: providing said ostomy pouch having a first fold line located at a top portion of said ostomy pouch, said ostomy pouch having a second fold line located at a bottom portion of said ostomy pouch, said bottom portion of said ostomy pouch is configured to be folded over at said second fold line and said top portion of said ostomy pouch is configured to be folded over at said first fold line

3. The method for bathing with a waterproof barrier covering an ostomy appliance of claim 2, further comprising the steps of: providing said ostomy pouch having a fastener connected to said top portion of said ostomy pouch, folding said bottom portion of said ostomy pouch at said second fold line, folding said top portion of said ostomy pouch at said first fold line, orienting said fastener of said top portion of said ostomy pouch over said bottom portion of said ostomy pouch, and fastening said top portion of said ostomy pouch to said bottom portion of said ostomy pouch.

4. (canceled)

5. The method for bathing with a waterproof barrier covering an ostomy appliance of claim 4, further comprising the steps of: inserting said ostomy pouch through said central opening of said removable plate, whereby, said ostomy pouch is retained within said chamber of said bowl.

6. The method for bathing with a waterproof barrier covering an ostomy appliance of claim 4, further comprising the steps of: depressing at least a portion of said removable plate at least one relief of said plurality of reliefs to allow clearance of said ostomy pouch through said central opening of said removable plate, whereby, said ostomy pouch is retained within said chamber of said bowl.
